(19) United States
(12) Patent Application Publication
(10) Pub. No.: US 2007/0282348 A1
(43) Pub. Date: Dec. 6, 2007

(54) Ophthalmic microsurgical instrument

(76) Inventor: Christopher F. Lumpkin, Steamboat Springs, CO (US)

Correspondence Address: Kenneth Watov, Esq.;WATOV & KIPNES, P.C., P.O. Box 247, Princeton Junction, NJ, 08550, US

(21) Appl. No.: 11/809,507

(22) Filed: Jun. 1, 2007

Related U.S. Application Data
(60) Provisional application No. 60/810,987, filed on Jun. 5, 2006.

(51) Int. Cl. A61F 9/00

(52) U.S. Cl. 606/107

(57) ABSTRACT

An ophthalmic microsurgical instrument for scraping or selectively removing tissue membrane from the retina, or other areas of the eye, is provided with a rod-like solid silicone tip, the front most portion of which is coated with a plurality of micron size sapphire chips providing an abrasive surface, both the angle and hardness of the silicone tip being determined for providing maximum tactile feel to a surgeon using the microsurgical instrument.

### RELATED APPLICATION

This application claims priority from U.S. Provisional Application No. 60/810,987, filed on Jun. 5, 2006, the content of which is incorporated herein by reference to the extent that it does not conflict herewith.

### FIELD OF THE INVENTION

The present invention relates broadly to ophthalmic microsurgical instruments, and more particularly relates to an ophthalmic microsurgical instrument adapted for selectively removing thin tissue portions from the retina and other areas of the eye.

### BACKGROUND OF THE INVENTION

Many instruments have been developed in the prior art for providing ophthalmic surgeons with instruments for removing proliferative membranes from the eye, particularly from the retina. There is a specific need in the art to further develop such instruments for providing better tactile feel to a surgeon when removing tissue from the retina, for example, or other areas of the eye. A number of such instruments include an elongated rod having attached to the free end thereof a hollow tip coated with a fine dust of hardened particles, such as diamond dust, for providing an abrasive surface. The tips are typically pointed. Such instruments typically include a handle from which the elongated rod projects, with the abrasive tip being located at the free end of the rod.

### SUMMARY OF THE INVENTION

The present invention relates to an ophthalmic microsurgical instrument adapted for selectively removing thin tissue portions from the retina and other areas of the eye. The microsurgical instrument of the present invention is especially useful for removing proliferative membranes in the treatment of proliferative vitreoretinal disorders. In one embodiment of the present invention, the microsurgical instrument includes a handle for grasping of the instrument by an ophthalmic surgeon, an elongated rigid tube, preferably stainless steel, having one end rigidly attached to the handle, and its other end secured to a solid resilient tip, preferably silicone. The silicone tip includes a pointed portion comprising a hardness value and an angle to the longitudinal axis, which optimizes the tactile feel of the instrument during use in the hands of the surgeon. Furthermore, in another embodiment, the microsurgical instrument of the present invention includes an adjustment mechanism for allowing the surgeon to adjust the length of the resilient tip extending from the instrument with greater ease and efficiency.

In one aspect of the present invention, there is provided an ophthalmic microsurgical instrument adapted for selectively removing thin tissue portions from the retina and other areas of the eye, comprising:

a handle having a free first end and an opposing second end;

a rigid first tube having one end secured to the second end of the handle, the other end of the tube being open;

a solid resilient rod having a portion of one end connected to the open end of said tube, the free end of the resilient rod comprising an angle relative to the longitudinal axis to form a sharpened and pointed tip, for providing maximum tactile feel to a surgeon using the instrument; and

an abrasive element secured to a forward most portion of the tip of said resilient rod.

In a further aspect of the present invention, there is provided an ophthalmic microsurgical instrument adapted for selectively removing thin tissue portions from the retina and other areas of the eye, comprising:

a handle having a first end, an opposing second end, and a cutout cavity being provided proximate the second end;

a rigid first tube having one end secured within a longitudinal axial hole running from the second end of the handle through to the cutout cavity within the handle, the other end of the tube being free;

a rigid second tube slideably contained within the first tube, having one end projecting into the cutout cavity of the handle, and another end protruding outward from the free end of the first tube;

a solid silicone rod having a portion securely installed in the second tube, a free end portion of the silicone rod comprising an angle relative to the longitudinal axis to form a sharpened and pointed tip protruding outward from the another end of said second tube, for providing maximum tactile feel to a surgeon using the instrument;

a plurality of micron size sapphire chips being secured to a forward most portion of the pointed tip of said silicone rod providing an abrasive tip thereon; and

a finger slide operatively associated with the cavity of the handle, with one end of the finger slide being secured to the end of the second tube projecting into the cavity, for selectively permitting the finger slide to be moved back and forth for moving the tip of the silicone rod to a desired position relative to the first tube.

### BRIEF DESCRIPTION OF THE DRAWING

The present invention is described with reference to the drawings, in which reference is made to specific dimensions, angles, and material for preferred embodiments, which embodiments are not meant to be limiting, wherein:

FIG. 1 is a right side elevational view of a surgical instrument for one embodiment of the present invention;

FIG. 2 is an exploded detailed view of a tip portion of the surgical instrument marked by circle A of FIG. 1 in accordance with the present invention;

FIG. 3 is a right side elevational view of a surgical instrument for a second embodiment of the present invention;

FIG. 4 is a top plan view of the surgical instrument of FIG. 3 in accordance with the present invention;

FIG. 5 is an exploded detailed view of a tip portion of the surgical instrument of FIG. 3 in accordance with the present invention;

FIG. 6 is a top plan view of a tip portion of a silicone rod for one embodiment of the present invention;

FIG. 7 is a left side elevational view of the tip portion of the silicone rod of FIG. 7 in accordance with the present invention;

FIG. 8 is a top plan view of the handle for the surgical instrument of FIG. 3 in accordance with the present invention;

FIGS. 9A and 9B are a flow diagram of the assembly process for the surgical instrument of FIG. 3 in accordance with the present invention;

FIG. 10 is a top plan view showing a portion of the assembly of a silicone rod inserted into a rigid tube for the embodiment of the invention of FIG. 3;

FIG. 11 is a top plan view of a fixture used for cutting an angular tip on a silicone rod; and

FIG. 12 is a left side elevational view of the fixture of FIG. 11, the right side elevational view being a mirror image thereof.

### DETAILED DESCRIPTION OF THE INVENTION

The present invention is directed to an ophthalmic microsurgical instrument adapted for selectively removing thin tissue portions from the retina and other areas of the eye. The microsurgical instrument of the present invention is especially useful for removing proliferative membranes in the treatment of proliferative vitreoretinal disorders. In one embodiment of the present invention, the microsurgical instrument includes a handle for grasping of the instrument by an ophthalmic surgeon, an elongated rigid tube, preferably stainless steel, having one end rigidly attached to the handle, and its other end secured to a solid resilient tip, preferably silicone. In another embodiment of the present invention, the tip can be the distal end of a silicone rod having its proximal end secured to the handle.

The silicone tip includes a pointed portion comprising a hardness value and an angle to the longitudinal axis, which optimizes the tactile feel of the instrument in the hands of the surgeon during use. In another embodiment, the microsurgical instrument of the present invention includes an adjustment mechanism for allowing the surgeon to adjust the length of the resilient tip extending from the instrument with greater ease and efficiency.

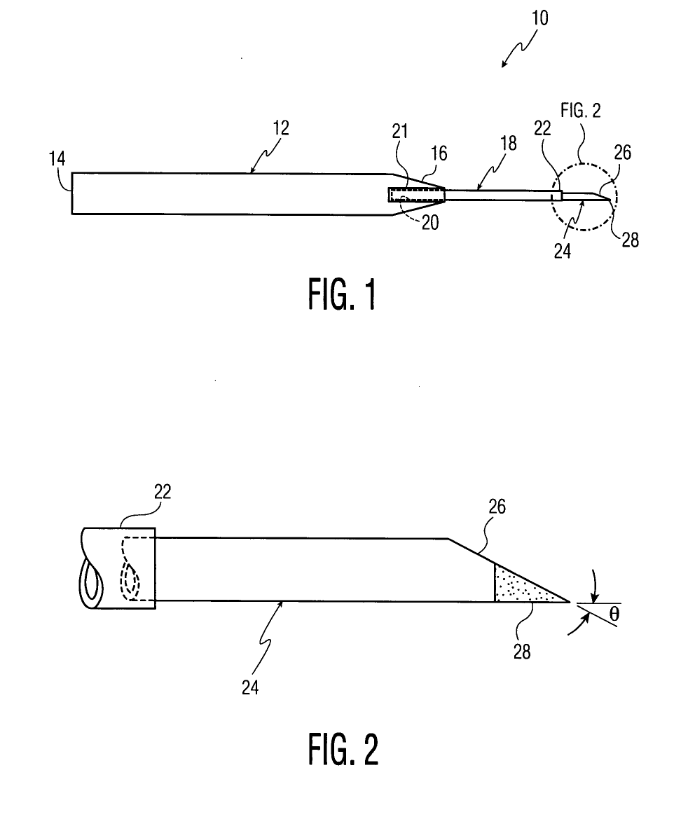

With reference to FIGS. 1 and 2, an ophthalmic microsurgical instrument identified generally by the reference numeral 10 is shown for one embodiment of the present invention. The microsurgical instrument 10 includes a handle 12 having a first end 14 and a second end 16 shaped for providing an ophthalmic surgeon with an area for gripping, an elongated rigid tube 18 having one end portion 20 secured via friction or a suitable adhesive such as, for example, cyanoacrylate, in a cylindrical cavity 21 in the second end 16. The remainder of the tube 18 extends away from the second end 16 of the handle 12. Lastly, a solid resilient rod 24 has one end portion retained within a distal end portion 22 of the tube 18. The solid resilient rod 24 further includes a tapered or pointed tip 26 supporting an abrasive element 28 for contacting the portions of the thin tissue to be removed from the retina and other areas of the eye.

The handle 12 is fabricated from a suitable rigid material such as, for example, wood, metal, plastic and the like. Preferably, the handle 12 is composed of lightweight, low-friction, and wear-resistant plastic including an acetal resin such as DELRIN®, marketed and sold by E.I. du Pont de Nemours and Company of Wilmington, Del. The tube 18 includes a proximal end 20 affixed to the second end 16 of the handle and an open distal end 22 in which a portion of the solid resilient rod 24 is securely retained therein by friction or via a suitable adhesive and/or wire. The tube 18 is selected from a needle gauge of from about 26 to 19 gauge, and preferably from about 25 to 20 gauge. The tube 18 is fabricated from a medically acceptable rigid material such as, metal, plastic, ceramic and the like, and preferably, a metal such as stainless steel or titanium.

The solid resilient rod 24 includes one end (not shown) secured and residing within the distal end 22 of the tube 18, and a free end in the form of a pointed tip 26, as previously described. The solid resilient rod 24 is maintained in a pliable and flexible state to allow the pointed tip 26 to conform and contact the abrasive element 28 to the surface of the tissue to be removed and minimize any unintentional damage to the surrounding healthy tissue of the retina and the eye, while providing proper tactile feel in the instrument 10 for the surgeon. The tip 26 of the solid resilient rod 24 is tapered or pointed, and can be formed by cutting the rod 24 at a bevel. The solid resilient rod 24 is fabricated from a medically acceptable flexible material such as, polymers, elastomers and rubbers, and preferably a polymer such as, polymerized siloxanes or silicone.

The solid resilient rod 24 exhibits a hardness value and an angle (at its tip 26), θ, relative to the longitudinal axis for providing maximum tactile feel to a surgeon using the instrument. The angle, θ, is in the range of from about 10° to 20°, preferably from about 14° to 18°, and more preferably 14°. The hardness value defined as the material's resistance to permanent indentation in polymers, elastomers and rubbers and is measured using a Shore durometer Type A scale. The hardness value of the solid resilient rod 24 is in the range of from about 25 to 70 A durometer, and preferably from about 36 to 50 A durometer.

Note that for each embodiment of the present invention, the angle, θ, shown for the tip 26 of the solid resilient rod 24, and the hardness thereof, have been experimentally determined to maximize the tactile feel to a surgeon using the associated microsurgical instrument, while at the same time optimizing the removal of tissue from the retina, or other areas of the eye. More specifically, the Inventor worked with physicians who used experimental prototypes having different tip angles to determine the angle for optimizing tactile feel.

The abrasive element 28 operates to abrade and remove the proliferative membranes on the retina with little or no damage to the surrounding healthy tissue. The abrasive element 28 is composed of a plurality of abrasive particles adhering to the tip 26 of the solid resilient rod 18. The abrasive particles are preferably composed of a rigid material of high hardness including, but not limited to, corundum, diamond, quartz, and the like. The abrasive particles can range in size of from about 10 microns to 40 microns, preferably 20 microns to 30 microns and more preferably 25 microns. In a preferred embodiment, the abrasive particles are in the form of 25-micron sapphire chips. The abrasive particles of the abrasive element 28 are attached to the tip 26 of the rod 24 through suitable adhesive materials such as medical grade room temperature vulcanization polymers including silicone. The method of attaching the abrasive particles can be implemented in any suitable manner as known in the art.

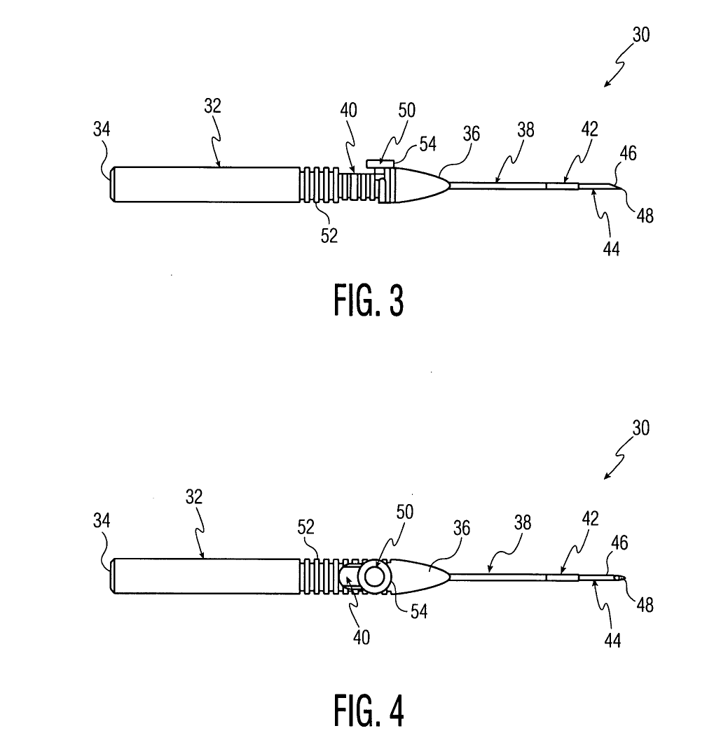

Referring to FIGS. 3-10, an ophthalmic microsurgical instrument identified generally by the reference numeral 30 is shown for a second embodiment of the present invention. The microsurgical instrument 30 includes a handle 32 having a first end 34 and a second end 36 configured for providing an ophthalmic surgeon with a secure grip, a cutout cavity 40 located proximate the second end 36 of the handle 32, and a series of gripping ribs 52 disposed proximate to the cutout cavity 40 for additional holding control.

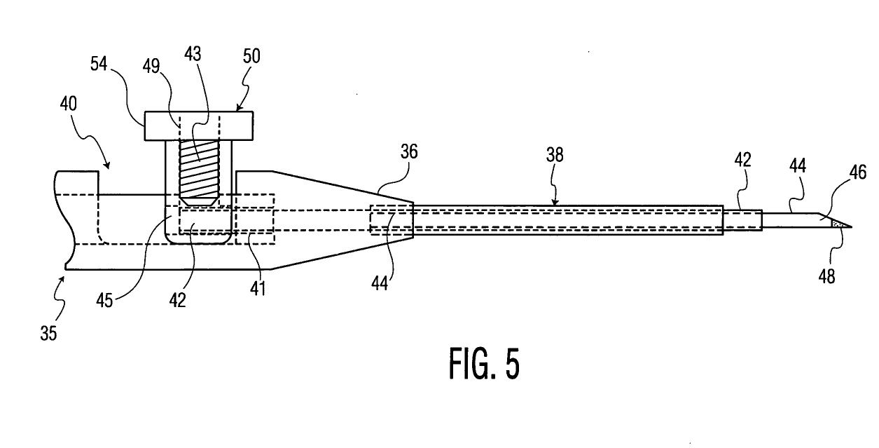

The microsurgical instrument 30 includes an elongated rigid first tube 38 extending away from the second end 36 of the handle 32, an elongated rigid second tube 42 telescopically engaged within the first tube 38 (as shown best in FIG. 5), and a solid resilient rod 44 having a portion securely contained within the second tube 42. The solid resilient rod 44 includes a relatively pointed tip 46 supporting an abrasive element 48 for contacting the portions of the thin tissue to be removed from the retina and other areas of the eye. The pointed tip 46 protrudes out of the end of the tube 42 remote from the handle 32.

The rigid second tube 42 includes one end projecting into the cutout cavity 40 of the handle 32, and another end protruding outward from the free end of the rigid first tube 38. The microsurgical instrument 30 also includes a finger slide 50 operatively associated with the cavity 50 of the handle 32. The finger slide 50 includes a finger-actuated slide button 54 at one end, and the other end is engaged to the end of the rigid second tube 42 projecting into the cavity 50. Typically, although not necessarily, the finger-actuated slide button 54 would be thumb actuated.

As shown in FIG. 5, the rigid first tube 38 encloses a major portion of the smaller diameter rigid second tube 42 in a telescopic arrangement. In this embodiment, the rigid second tube 42 is adapted to securely retain a major portion of the solid resilient rod 44. The distal end of the rod 44 providing the tip 46 extends away from the second tube 42. The second tube 42 containing the rod 44 is moveable using the finger slide 50 via the slide button 54 at the upper portion of the handle 32. In this manner, the slide button 54 can be moved back and forth from one position to another to allow the surgeon to selectively adjust the desired position of the rod tip 46 relative to the rigid first tube 38. This allows the tip 46 of the silicone rod 44 to be retractable as needed to protect the abrasive element 48 from damage during non-use. As shown in FIG. 5, a tubular sleeve 41 secured near the interior end of and on the tube 42 is positioned in a lateral hole 45 of the slid button 54, and secured thereto via a set screw 43 mounted on an axial threaded hole 49 in the button 54.

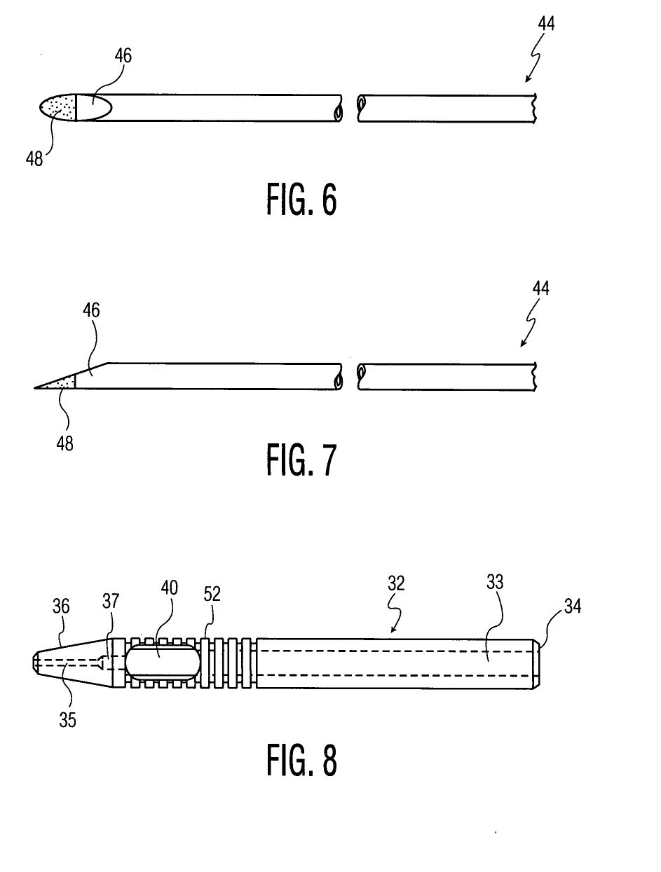

Referring to FIGS. 6 and 7, the solid resilient rod 44 is shown with the tapered or beveled tip 46, and the area supporting the abrasive element 48. Also, with reference to FIG. 8, note that the tapered end 36 of handle 32 includes a centrally located hole 35 for receiving the rigid first tube 38. Following hole 35 is a larger diameter hole 37 for receiving a portion of sleeve 41, if necessary.

### EXAMPLES

### Example 1

### Procedure for Assembling an Ophthalmic Microsurgical Instrument

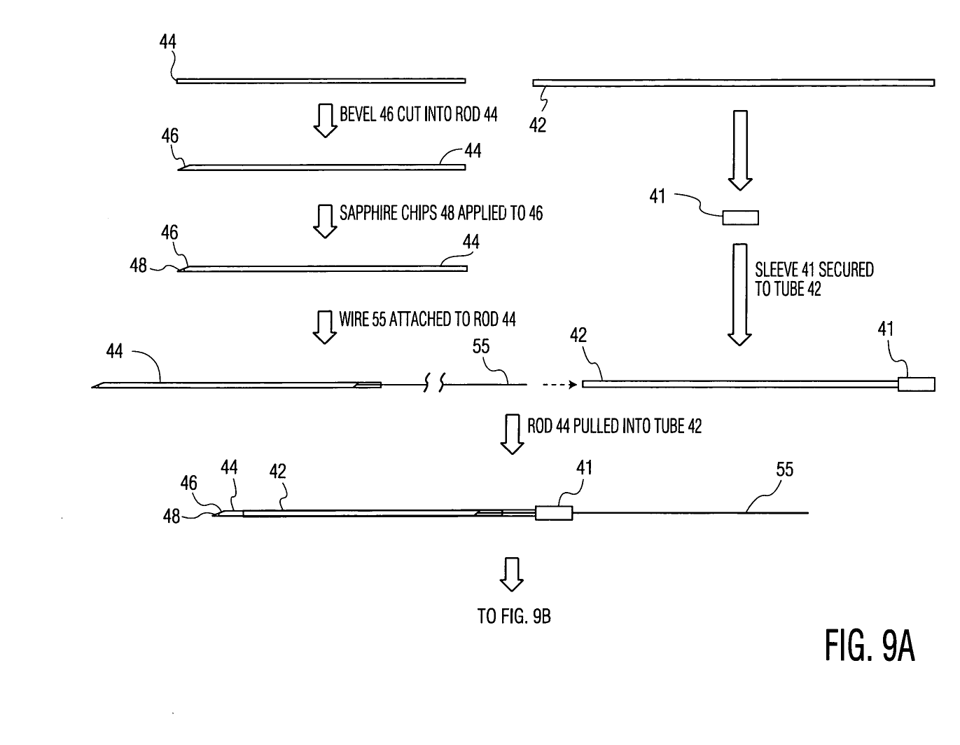

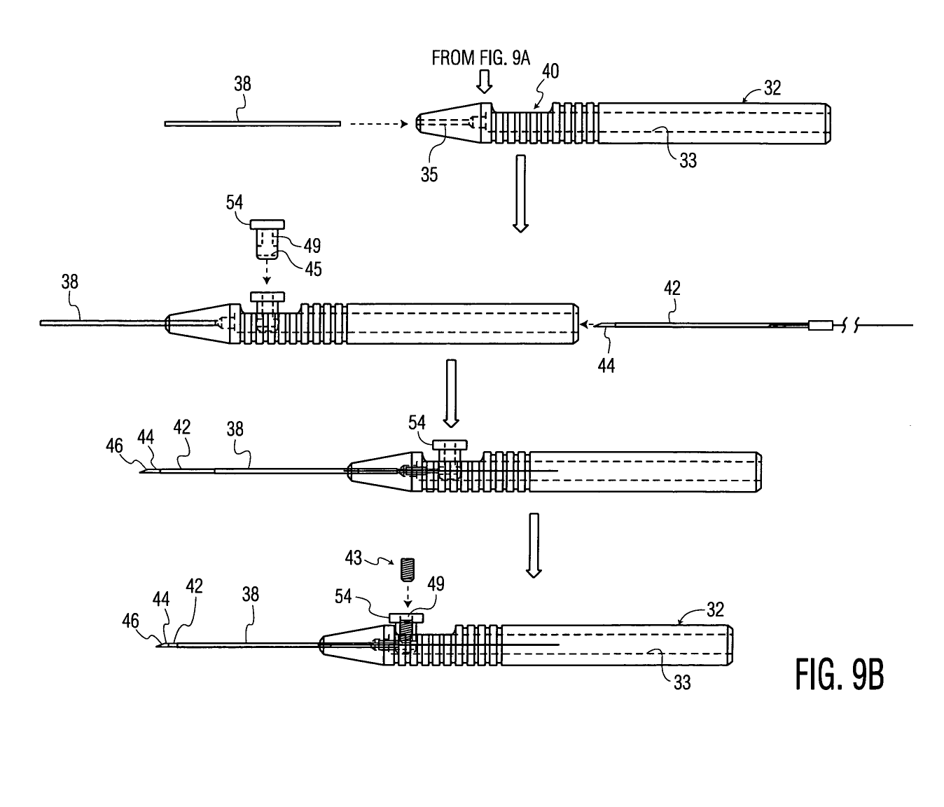

Purpose & Scope: To describe and document the procedure for assembling an ophthalmic microsurgical instrument, for example, with reference to FIGS. 9A and 9B through 12.

### Materials and Equipment:

### 1) Razor

### 2) Wicking Adhesive (cyanoacrylate, for example)

### 3) Lint Free Wipe

### 4) Alcohol (70% isopropyl)

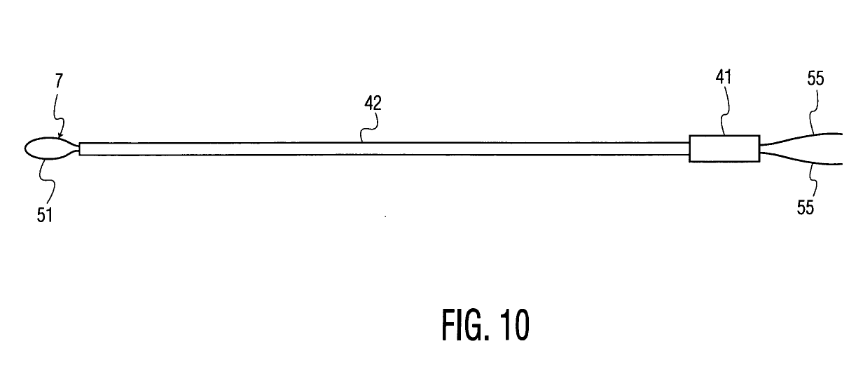

As shown in FIGS. 9A and 9B, the tube 38 was pressed into a handpiece 32 leaving 1.181 inches exposed. The tubular sleeve 41 was adhered to the tube 42 as shown using a suitable adhesive. In this example, the sleeve 41 was 20-gauge XX wall, 0.25″. The rigid second tube 42 is 22-gauge XX wall, 2.05″, for example. A 5″ segment of a spring wire 7 (see FIGS. 9A and 9B and 10) was cut, and inserted into the 20-gauge/22-gauge subassembly of sleeve 41 and the second tube 42. The end of the spring wire 7 was passed through the subassembly 41, 42, and looped back into the subassembly 41, 42 until the two free ends 55 of the spring wire 7 are even with one another.

The two ends 55 of the spring wire 7 were pulled back until the end of the loop 51 at the opposite end of the subassembly 41, 42 was approximately ⅛″ in length, for example. A silicone rod 44 was obtained from a holder (not shown). With the end holding the sapphire chips 48 first, approximately two-thirds of the rod 44 was inserted into the loop 51 of the spring wire 7. The ends 55 of the spring wire 7 were pulled back, which urged the loop 51 and the silicone rod 44 into the 20-gauge/22-gauge subassembly 41, 42. The ends 55 of the spring wire 7 were pulled back until 0.063″ of the silicone rod 44 is extending beyond the end of the rigid second tube 42 (22-gauge needle), for example.

A small amount of a wicking adhesive was applied to the end of the needle subassembly 41, 42 that does not have the silicone rod 44. After the application of the wicking adhesive, the ends 51 of the spring wire 7 were trimmed flush with the needle subassembly 41, 42. The needle subassembly 41, 42 was threaded into the proximal end of the handpiece 32 with the sapphire chips 48 inserted first. Once the silicone rod 44 cannot be inserted any further into the handpiece 32 (i.e., when the rod 44 abuts with the first tube 38 (20-gauge needle)), the needle subassembly 41, 42 was gently retracted enough to insert the finger slide 50 into the slotted handle 32, using a pair of forceps (not shown). The silicone rod 44 was inserted into the through hole 45 of the finger slide 50, and the silicone rod 44 was extended into the first tube 38 (20-gauge needle) that was pressed into the handpiece 32, with the help of the forceps.

With the finger slide 50 positioned to the proximal end of the slot 40 in the handle 32, the silicone rod 44 was extended until it is flush with the end of the first tube 38 (20-gauge needle). A set screw 43 was inserted into the button 54 and finger slide 50, and advanced down onto the needle subassembly 41, 42. The set screw 43 was sufficiently torqued so that it was securely seated onto the sleeve 41 of the needle subassembly 41, 42.

The finger slide 50 was pushed forward so that the silicone rod 44 is fully extended. The set screw 43 was slightly unscrewed so that the second tube 42 (22-gauge needle) and the silicone rod 44 can be rotated. The second tube 42 (22-gauge needle) was rotated until the angle of the silicone rod 44 is positioned 180 degrees from the finger slide 50 (see FIG. 5). At this point, the set screw 43 was firmly tightened. The silicone rod 44 was inserted into an ultrasound cleaning formulation consisting of a 50/50 solution of water and alcohol, and submerged for 30 seconds. The silicone rod 44 was gently blow dried with 25 psi air pressure.

A 100% inspection was performed in accordance with ITP80-OL. The handpiece 32 was wiped with lint free wipe moistened with alcohol. The wipe was made from the end of the handpiece 32 closest to the needle subassembly 41, 42, and moved back toward the opposite end of the handpiece 32.

### Example 2

### Procedure for Producing Silicone Strips as Starting Material for the Fabrication of Silicone Rods for the Membrane Scraper

Purpose & Scope: To describe and document the procedure for creating a silicone strip used in the fabrication of silicone rods with sapphire chips forming part of the assembly of the ophthalmic microsurgical instrument procedure of Example 1.

### Materials and Equipment:

### 1) Release Agent and Squirt Bottle

### 2) Oven

### 3) Cookie Sheet (12″×12″ round)

### 4) Scale or Cutting Template

### 5) Razor

### 6) Holding Cup

A polyimide tubing was cut into 0.75″ lengths using a razor and cutting template to yield about 10 to 15 polyimide tubes. A release agent was sprayed into the interior of the polyimide tubes. Compressed air at 80 psi was directed into the polyimide tubes to expel any excess release agent therefrom. About 10 cc of silicone were prepared and loaded into a syringe, ensuring that all bubbles are excluded. The silicone was then injected from the syringe into each of the polyimide tubes until a small amount exits the opposite distal end. The filled polyimide tubes were arranged on a cookie sheet. The cookie sheet was placed into an oven preheated to about 350° F. for about 2 hours. After 2 hours, the silicone strips formed within the polyimide tubes were then extracted by firmly pulling on the remnant of silicone extending from the end of the tube. The silicone strips were then stored in a holding cup.

### Example 3

### Procedure for Fabricating a Silicone Rod with Sapphire Chips

Purpose & Scope: To describe and document the procedure for fabricating a silicone rod with sapphire chips forming part of the assembly of the ophthalmic microsurgical instrument procedure of Example 1.

### Materials and Equipment:

### 1) Oven

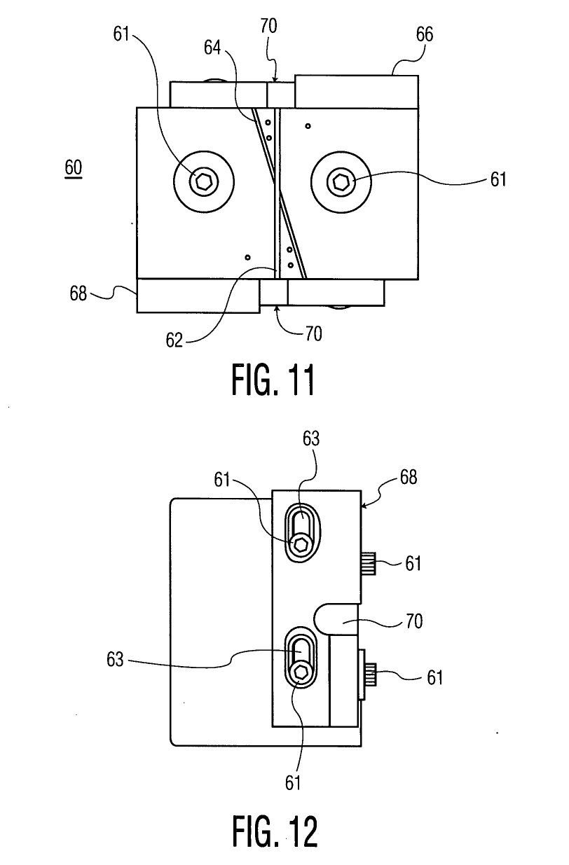

### 2) Fixture (see FIGS. 11 and 12)

### 3) Forceps

### 4) Razor

### 5) Silicone Adhesive

A length of silicone strip was uncoiled, and laid flat on a work surface. The silicone strip was cut into manageable portions and retained in a holding cup (not shown). A single silicone strip portion was obtained from the holding cup (not shown) and placed onto a fixture 60 along a grove 62, with an end portion of the silicone strip positioned beyond a razor blade groove 64. A razor blade was run through the groove 64 in order to produce a desired cut angle. This yielded two silicone rods 44, if one rod 44 of sufficient length is centered on either side of the razor groove 64, while being retained in the groove 62. Note that before cutting, the silicone strip is preferably stretched by loosening the set screws 61 of either or on each side piece member 66 and 68 to move them in a direction to slightly bend the end portions of the silicone strip, whereafter the set screws 61 are tightened. In this manner, the silicone strip is tightly stretched to provide a cleaner cut end, which forms the tip 46 of the silicone rod 44.

Using forceps, the silicone rods 44 were dipped into a solution of 50% alcohol. Each of the silicone rods 44 was blow dried, and placed in a hold cup (not shown) with the cut or pointed tip 46 exposed. A dab of silicone adhesive was placed on the side of a razor blade. This is the correct amount due to the fact that the adhesive cures on its own and in the time it takes to apply the adhesive, the adhesive may begin to become unworkable. Under a microscope, the adhesive precisely applied to both sides of the pointed tip 46 of a silicone rod 44. The adhesive should not be applied to any area more than one half of the way down the silicone rod 44. Also, the adhesive should be applied thinly. A bulb or bubble will cause an unwanted amount of sapphire chips 48 to adhere to the silicone. A teaspoon amount of sapphire chips 48 was placed onto a clean work surface.

The silicone rod 44 with the adhesive was lightly brushed back and forth into the sapphire chips 48, and ensured that the entire surface of the exposed adhesive is covered. Any excess sapphire chips 48 were gently knocked off through tapping of the silicone rod 44 against the side of the container holding the sapphire chips 48. The silicone rod 44 with the adhered sapphire chips 48 was placed into a holding cup (not shown), and cured in an oven at a temperature of about 300° F. for about 2 hours. Using forceps, the silicone rod 44 was removed from the holding cup, and placed into an ultrasound cleaning formulation containing a mixture of 50% water and 50% alcohol, for about 30 seconds. The silicone rod 44 was blow dried, and then place back into the holding cup.

Although various embodiments of the invention are shown and described, they are not meant to be limiting. Those of skill in the art may recognize certain modifications to those embodiments, which modifications are meant to be covered by the spirit and scope of the appended claims. For example, the wire gauges, tubular member sizes, silicone rod sizes, tip angles, and other dimensions provided above are not meant to be limiting.

What is claimed is:

1. An ophthalmic microsurgical instrument adapted for selectively removing thin tissue portions from the retina and other areas of the eye, comprising:
a handle having a free first end and an opposing second end;
a rigid first tube having one end secured to the second end of the handle, the other end of the tube being open;
a solid resilient rod having a portion of one end connected to the open end of said tube, the free end of the resilient rod comprising an angle relative to the longitudinal axis to form a sharpened and pointed tip, for providing maximum tactile feel to a surgeon using the instrument; and
an abrasive element secured to a forward most portion of the tip of said resilient rod.

2. The microsurgical instrument of claim 1, wherein the rigid first tube is stainless steel.

3. The microsurgical instrument of claim 1, wherein the solid resilient rod is a material selected from the group consisting of polymers, elastomers, rubbers and combinations thereof.

4. The microsurgical instrument of claim 3, wherein the polymer is silicone.

5. The microsurgical instrument of claim 1, wherein the angle is in the range of from about 10° to 20°.

6. The microsurgical instrument of claim 5, wherein the angle is 14° to 18°.

7. The microsurgical instrument of claim 6, wherein the angle is 14°.

8. The microsurgical instrument of claim 1, further including a predetermined hardness for the resilient rod for maximizing the tactile feel of the user.

9. The microsurgical instrument of claim 8, wherein the hardness is in the range of from about 25 to 70 A durometer.

10. The microsurgical instrument of claim 9, wherein the hardness is in the range of from about 36 to 50 A durometer.

11. The microsurgical instrument of claim 1, wherein the abrasive element comprises a plurality of abrasive particles.

12. The microsurgical instrument of claim 11, wherein the abrasive particles are selected from the group consisting of corundum, diamond, quartz, and combinations thereof.

13. The microsurgical instrument of claim 12, wherein the corundum comprises micron sized sapphire chips.

14. The microsurgical instrument of claim 1, further comprising:
a cutout cavity being provided proximate the second end of the handle;
a second rigid tube slideably contained within the rigid first tube, having one end projecting into the cutout cavity of the handle, and another end protruding outward from the free end of the rigid first tube;
said solid resilient rod having said portion of its one end rigidly secured within said another end of said second rigid tube, thereby providing the connection between said first tube and said resilient rod; and
a finger slide operatively associated with the cavity of the handle, with one end of the finger slide being secured to the end of the second rigid tube projecting into the cavity, for selectively permitting the finger slide to be moved back and forth for moving the tip of the resilient rod to a desired position relative to the rigid first tube.

15. An ophthalmic microsurgical instrument adapted for selectively removing thin tissue portions from the retina and other areas of the eye, comprising:
a handle having a first end, an opposing second end, and a cutout cavity being provided proximate the second end;
a rigid first tube having one end secured within a longitudinal axial hole running from the second end of the handle through to the cutout cavity within the handle, the other end of the tube being free;
a rigid second tube slideably contained within the first tube, having one end projecting into the cutout cavity of the handle, and another end protruding outward from the free end of the first tube;
a solid silicone rod having a portion securely installed in the second tube, a free end portion of the silicone rod comprising an angle relative to the longitudinal axis to form a sharpened and pointed tip protruding outward from the another end of said second tube, for providing maximum tactile feel to a surgeon using the instrument;
a plurality of micron size sapphire chips being secured to a forward most portion of the pointed tip of said silicone rod providing an abrasive tip thereon; and
a finger slide operatively associated with the cavity of the handle, with one end of the finger slide being secured to the end of the second tube projecting into the cavity, for selectively permitting the finger slide to be moved back and forth for moving the tip of the silicone rod to a desired position relative to the first tube.

16. The microsurgical instrument of claim 15, wherein the angle is in the range of from about 10° to 20°.

17. The microsurgical instrument of claim 16, wherein the angle is 140 to 180.

18. The microsurgical instrument of claim 15, further including a predetermined hardness for the resilient rod for maximizing the tactile feel of the user.

19. The microsurgical instrument of claim 18, wherein the hardness is in the range of from about 25 to 70 A durometer.

20. The microsurgical instrument of claim 19, wherein the hardness is in the range of from about 36 to 50 A durometer.

21. The microsurgical instrument of claim 15, wherein the first and second tubes each comprises stainless steel.
